(19) United States
(12) Patent Application Publication
(10) Pub. No.: US 2018/0070836 A1
(43) Pub. Date: Mar. 15, 2018

(54) CVHR SHAPE MEASUREMENT DEVICES

(71) Applicants: Public University Corporation Nagoya City University, Aichi (JP); SUZUKEN CO., LTD., Aichi (JP)

(72) Inventor: Junichiro HAYANO, Aichi-ken (JP)

(73) Assignees: Public University Corporation Nagoya City University, Aichi (JP); SUZUKEN CO., LTD., Aichi (JP)

(21) Appl. No.: 15/557,778

(22) PCT Filed: Jan. 5, 2016
(86) PCT No.: PCT/JP2016/050154

§ 371 Date: Sep. 12, 2017

(30) Foreign Application Priority Data
Mar. 12, 2015 (JP) 2015-049863

(51) Int. Cl. A61B 5/0245; A61B 5/08

(52) U.S. Cl. CPC A61B 5/0245; A61B 5/08

(57) ABSTRACT

In some embodiments, a device to measure a shape of cyclic variation of heart rate (CVHR) comprises a processor to detect CVHR from time series data of cardiac or pulse wave inter-beat interval, heart rate, or pulse rate; acquire at least one shape property index with respect to a waveform of the detected CVHR; and evaluate health risk based on the at least one shape property index. The CVHR appears periodically in the time series data. The at least one shape property index is selected from the group consisting of an amplitude of the CVHR (ACV), a slope, a ratio of the ACV to a duration, and an area. The health risk is inversely correlated with an intensity of a heart rate response determined from the at least one shape property index.

### CROSS-REFERENCE TO RELATED APPLICATIONS

The present application is a National Phase entry of, and claims priority to, PCT Application No. PCT/JP2016/050154, filed Jan. 5, 2016, which claims priority to Japanese Patent Application No. 2015-049863, filed Mar. 12, 2015, both of which are incorporated by reference herein in their entireties for all purposes.

### STATEMENT REGARDING FEDERALLY SPONSORED RESEARCH OR DEVELOPMENT

Not applicable.

### BACKGROUND

Japanese Patent Application Publication No. 2010-51387 describes a technique that detects CVHR accompanying apnoeic episodes or hypopnoeic episodes of sleep disordered breathing. In Japanese Patent Application Publication No. 2010-51387, the technique is used to measure a subject's frequency of CVHR (frequency of cyclic variation, FCV) per unit time (which is one hour in the Publication), which may detect whether or not the subject is affected by obstructive sleep apnea syndrome (OSAS).

### BRIEF DESCRIPTION OF THE DRAWINGS

FIG. 1 is a block diagram showing a configuration of a CVHR shape measurement device;

FIG. 2 is a diagram showing R-R interval time series data for 24 hours measured by a Holter electrocardiograph;

FIG. 3(a) shows a graph A showing ACV logarithmic distributions for respective FCV logarithms of groups having same FCV logarithms, FIG. 3(b) shows a graph B showing a relationship between the FCV logarithms and ACV logarithmic means in the graph of FIG. 3(a), and FIG. 3(c) shows a graph C showing a relationship between the FCV logarithms and ACV logarithmic standard deviations in the graph of FIG. 3(a);

FIG. 4 is a flowchart of an ACV score measurement process;

FIG. 5 is a flowchart in continuation of FIG. 4;

FIG. 6 is a flowchart in continuation of FIG. 5;

FIG. 7 is a flowchart of a dip depth calculation process;

FIG. 8 is a flowchart of a logarithm calculation process for an amplitude of CVHR (ACV);

FIG. 9 is a flowchart of an ACV score calculation process;

FIG. 10 schematically shows R-R interval time series data;

FIG. 11 schematically shows the R-R interval time series data;

FIG. 12(a) shows a state in which n sets of CVHR detected by a CVHR detection means are segmented into n sets of segments s1 to sn, and FIG. 12(b) shows a mean time series created by averaging the n sets of segments s1 to sn;

FIG. 13(a) shows a graph D of a mean time series of a subject with good prognosis, and FIG. 13(b) shows a graph E of a mean time series of a subject who passed away after one year;

FIG. 14(a) shows Kaplan-Meier curves of ACV scores and mortality of a group affected with acute myocardial infarction;

FIG. 14(b) shows Kaplan-Meier curves of ACV scores and mortality of another group affected with acute myocardial infarction;

FIG. 14(c) shows Kaplan-Meier curves of ACV scores and mortality of a group of end-stage renal failure patients receiving hemodialysis;

FIG. 14(d) shows Kaplan-Meier curves of ACV scores and mortality of a group affected with chronic heart failure; and

FIG. 15 is a diagram for explaining respective shape property indexes of CVHR.

### DETAILED DESCRIPTION

In the technique of Japanese Patent Application Publication No. 2010-51387, the CVHR is detected and the FCV thereof is measured to predict whether or not the subject is affected by sleep apnea syndrome, and to estimate its severity. However, as a result of the study conducted by the inventor, it has been revealed that a degree of the FCV is not so relevant to a degree of health risk such as mortality within a predetermined period for the subject (hereinbelow referred to as the health risk). That is, it has been revealed that the FCV is insufficient as an index for predicting such health risk.

In the present description, a technique that can more accurately predict a degree of health risk such as mortality within a predetermined period is disclosed.

A cyclic variation of heart rate (CVHR) shape measurement device disclosed herein comprises a CVHR detection means and a CVHR shape property index acquisition means. The CVHR detection means is configured to detect CVHR from data indicating cycles or frequency of heart rate or pulse in time series. The CVHR shape property index acquisition means is configured to acquire at least one of the following shape property indexes with respect to a waveform of the CVHR detected by the CVHR detection means: an amplitude of cyclic variation (ACV), a slope, a ratio of the ACV to a duration, and an area. It should be noted that the pulse rate is cooperative with the heart rate. Due to this, a cyclic variation of pulse rate herein will also be collectively included in “cyclic variation of heart rate (CVHR)”.

Further, CVHR is generally defined as “a variation in the heart rate that cyclically appears accompanying apnoeic episode or hypopnoeic episode during sleep”, however, the CVHR herein is broadly defined as “a variation in the heart rate accompanying apnea or hypopnea”. That is, in this description, the variation in the heart rate accompanying the apnea or hypopnea not only during sleep but also during a waking period is included in the broadly-defined CVHR as a CVHR-related phenomenon. Due to this, the CVHR herein is not limited to those appearing accompanying the apnoeic episodes or hypopnoeic episodes caused due to sleep disordered breathing.

Further, “apnea or hypopnea” described herein is not limited to those naturally occurring due to episodes, but also includes artificially-caused ones, for example, by intentionally stopping breathing or reducing respiration volume during the waking period. A heart rate variation caused thereby will be also included in the broadly-defined CVHR as the CVHR-related phenomenon. Further, in this description, any heart rate variation generated by apnea or hypopnea will be included in the broadly-defined CVHR as CVHR-related phenomena, even if it does not exhibit a cyclic pattern. In other words, in this description, a physiological heart rate variation (that is, heart rate variation other than the heart rate variation occurring accompanying apnea or hypopnea) is not included in the CVHR.

Here, each of the shape property indexes of the CVHR will be described. Each CVHR waveform has a first maximal value, a minimal value, and a second maximal value. The first maximal value is a point that appears before the minimal value in time series and is the closest to the minimal value, and the second maximal value is a point that appears after the minimal value in the time series and is the closest to the minimal value. The amplitude of cyclic variation (ACV) is a distance between the minimal value and a straight line connecting the first maximal value and the second maximal value. The slope is found by dividing the amplitude of cyclic variation by an elapsed time from the first maximal value to the minimal value, and/or found by dividing the amplitude of cyclic variation by an elapsed time from the minimal value to the second maximal value. The duration is an elapsed time from the first maximal value to the second maximal value. Due to this, the ratio of the amplitude of cyclic variation to the duration is found by dividing the distance between the minimal value and the straight line connecting the first maximal value and the second maximal value by the elapsed time from the first maximal value to the second maximal value. The area is a size of an area defined by the CVHR waveform and the straight line connecting the first maximal value and the second maximal value. It should be noted that “one CVHR” may refer to CVHR obtained from one heart rate variation, or may be CVHR obtained by averaging multiple instances of heart rate variations. It should be noted that the CVHR shape property index acquisition means may acquire not only the amplitude of cyclic variation (ACV), but also an ACV-related property (for example, a logarithm thereof).

The CVHR is the heart rate variation accompanying apnea or hypopnea. Due to this, the amplitude of cyclic variation (ACV) of the CVHR can be said to indicate an intensity of heart rate response to apnoeic load or hypopnoeic load. Further, the other shape property indexes (the slope, the ratio of the amplitude of cyclic variation to the duration, and the area of the CVHR) are values related to the intensity of the heart rate response to the apnoeic load or hypopnoeic load. As a result of the keen study conducted by the inventor, it has been revealed that a magnitude of the ACV (that is, the intensity of the heart rate response to the apnoeic load or hypopnoeic load) and a degree of health risk such as mortality within a predetermined period (hereinbelow referred to as the health risk) are deeply related to each other, and that the ACV can be a useful index in predicting such health risk. Specifically, it has been revealed that the health risk is further reduced with a larger ACV (that is, with stronger heart rate response to the apnoeic load or hypopnoeic load), and the health risk is further increased with a smaller ACV (that is, with weaker heart rate response to the apnoeic load or hypopnoeic load). Further, it has been revealed that the other shape property indexes (the slope, the ratio of the amplitude of cyclic variation to the duration, and the area of the CVHR) can be useful indexes for predicting the health risk as well. Specifically, the health risk is further reduced with a larger slope (absolute value thereof) of the CVHR. Further, the health risk is further reduced with a greater ratio of the amplitude of cyclic variation to the duration of the CVHR. Further, the health risk is further reduced with a larger area of the CVHR. In the aforementioned CVHR shape measurement device, the CVHR detection means detects the CVHR, and the CVHR shape property index acquisition means acquires at least one of the shape property indexes, which are the amplitude of cyclic variation (ACV), the slope, the ratio of the amplitude of cyclic variation to the duration, and the area of the CVHR. The CVHR shape measurement device can predict the health risk more accurately than conventional techniques by referencing at least one of the shape property indexes acquired by the CVHR shape property index acquisition means. It should be noted that the health risk may include, other than mortality within a predetermined period as aforementioned, morbidity rate, incidence rate, recurrence rate (rate of rehospitalization), degree of disease progression, and the like.

Further, the present description discloses another novel cyclic variation of heart rate (CVHR) shape measurement device configured to be able to solve the aforementioned problem. This CVHR shape measurement device comprises a CVHR input means and a CVHR shape property index acquisition means. The CVHR input means is configured to input periodically appearing CVHR specified from data indicating cycles or frequency of heart rate or pulse rate in time series. The CVHR shape property index acquisition means is configured to acquire at least one of the following shape property indexes with respect to a waveform of the CVHR inputted by the CVHR input means: an amplitude of cyclic variation (ACV), a slope, a ratio of the ACV to a duration, and an area. This CVHR shape measurement device does not detect the CVHR. That is, CVHR that had been detected outside the device is inputted to this CVHR shape measurement device. Due to this, the CVHR shape measurement device can be used by being connected to various types of CVHR-detectable devices.

Further, the present description discloses executable code which can solve the aforementioned problem. This code causes a computer to perform a CVHR detection process and a CVHR shape property index acquisition process. In the CVHR detection process, CVHR is detected from data indicating cycles or frequency of heart rate or pulse rate in time series. In the CVHR shape property index acquisition process, at least one of the following shape property indexes with respect to a waveform of the CVHR detected in the CVHR detection process is acquired: an amplitude of cyclic variation (ACV), a slope, a ratio of the ACV to a duration, and an area. By using this executable code, a CVHR shape measurement device capable of predicting a degree of the health risk such as mortality within a predetermined period more accurately than conventional techniques can be realized.

Further, the present description discloses another novel cyclic variation of heart rate (CVHR) shape measurement device configured to be able to solve the aforementioned problem. This CVHR shape measurement device comprises a CVHR detection means, a CVHR shape acquisition means, and an evaluation means. The CVHR detection means is configured to detect CVHR from data indicating cycles or frequency of heart rate or pulse rate in time series. The CVHR shape acquisition means is configured to acquire a shape of a waveform of the CVHR detected by the CVHR detection means. The evaluation means is configured to evaluate health risk only based on the shape of the CVHR obtained by the CVHR shape acquisition means. According to this CVHR shape measurement device, a degree of the health risk can easily be recognized.

Some of the features characteristic to the below-described embodiments will herein be listed. It should be noted that the respective technical elements are independent of one another, and are useful solely or in combination. The combinations thereof are not limited to those described in the claims as originally filed.

In a CVHR shape measurement device disclosed herein, a CVHR shape property index acquisition means may be configured to acquire at least one of an ACV, a slope, a ratio of the ACV to a duration, and an area by averaging data indicating respective waveforms of a plurality of CVHR detected during a predetermined period of the data. Shapes (waveforms) of the CVHR vary according to a degree of respiration (being apnoeic or hypopnoeic), or according to a duration of apnea or hypopnea, or the like, and thus, there is variation in each shape property index among the CVHR. Due to this, by averaging the waveforms of the plurality of CVHR and acquiring each of the shape property indexes of one CVHR generated by the averaging, the reliability of each shape property index can be increased.

The CVHR shape measurement device disclosed herein may further comprise an FCV acquisition means and an ACV correction means. The FCV acquisition means may be configured to acquire a frequency of the CVHR (FCV) per unit time which were detected by the CVHR detection means during the predetermined period. The ACV correction means may be configured to acquire a corrected ACV (ACV score) by correcting the ACV based on a value of the FCV. According to the study by the inventor, the ACV and the FCV are correlated. Due to this, by correcting the ACV based on the value of the FCV, the corrected ACV (ACV score), which is the ACV that has been corrected, functions as a versatile index independent from the value of the FCV. Thus, health risk can more accurately be predicted. It should be noted that the FCV acquisition means may measure not only the FCV but also a property related to the FCV.

Further, the CVHR shape measurement device disclosed herein can measure, if the CVHR occurs just even once, the amplitude of that CVHR (ACV). That is, the CVHR needs to appear once in the data. If the period of the data exceeds the unit time, the FCV needs to be greater than zero (for example, in a case where the data period is two hours and the unit time is one hour, if one CVHR appears in the data, the FCV becomes 0.5).

In the CVHR shape measurement device disclosed herein, the ACV correction means may be configured to correct the ACV by using the following two functions which are derived from a database storing a plurality of sets of associations of an ACV acquired from the data during the predetermined period and an FCV acquired from the data during the predetermined period: an average function of the ACV which is a function of the FCV and a standard deviation function of the ACV which is a function of the FCV; and to correct the ACV acquired by the CVHR shape property index acquisition means using a mean acquired from the average function and a standard deviation acquired from the standard deviation function which correspond to an FCV acquired from the data during the predetermined period from which the ACV is acquired. It should be noted that the “mean of the ACV” herein means a mean of ACVs of plural subjects having a same FCV. According to this configuration, the corrected ACV (ACV score) can be calculated as one type of deviation value of the ACV. By using the functions derived from the database for the correction, versatility of the ACV score can be improved. It should be noted that the aforementioned two functions may respectively be an FCV logarithm, an ACV logarithmic mean, and an ACV logarithmic standard deviation.

In the CVHR shape measurement device disclosed herein, the data may indicate any one of an R-R interval, a pulse rate interval, and a heart rate interval in time series. Such data can easily be acquired using currently-popularized devices (e.g., a Holter electrocardiograph, a wearable plethysmograph, or an ictometer). Due to this, there is no need for hospitalization to acquire the data, and the data can easily be acquired. By using the aforementioned CVHR shape measurement device, the ACV values can be measured non-invasively, safely, and repeatedly in daily life. Due to this, the CVHR shape measurement device can be used as a tool for managing a user's own health.

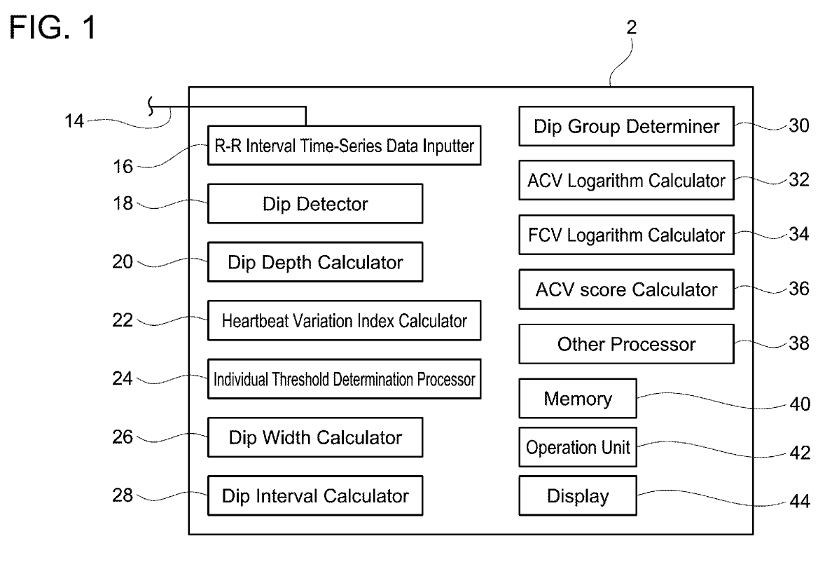

An embodiment will be described with reference to the drawings. FIG. 1 is a block diagram showing a configuration of a CVHR shape measurement device 2 of the present embodiment. The CVHR shape measurement device 2 comprises an R-R interval time series data inputter 16, a dip detector 18, a dip depth calculator 20, a heart rate variation index calculator 22, an individual threshold determination processor 24, a dip width calculator 26, a dip interval calculator 28, a dip group determiner 30, an ACV logarithm calculator 32, an FCV logarithm calculator 34, an ACV score calculator 36, other processor 38, a memory 40, an operation unit 42, and a display 44. It should be noted that the respective modules 16 to 38 are implemented by a computer installed in the CVHR shape measurement device 2 executing processes according to executable code.

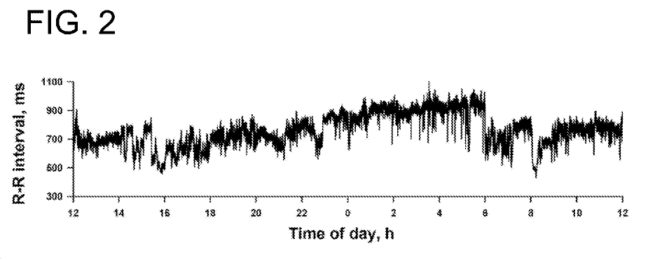

The R-R interval time series data inputter 16 is connected to a communication line 14. The communication line 14 is connected to an R-R interval measuring device (which is a Holter electrocardiograph in the present embodiment). The R-R interval time series data inputter 16 inputs R-R interval time series data of a human, which was measured and outputted by the R-R interval measuring device. FIG. 2 shows an example of the R-R interval time series data. In FIG. 2, the R-R interval time series data is measured over twenty-four hours. The dip detector 18 detects a plurality of local dips from the R-R interval time series data. In the present embodiment, the dip detector 18 detects the plurality of local dips from the R-R time series data during time in bed of the twenty-four-hour R-R interval time series data. It should be noted that the time in bed means a time range during which a subject is in bed, and it should be noted that the subject may be awake. Further, the time in bed may be specified according to the subject's report, or for example, seven hours from 11 p.m. to 6 a.m. on the next day may be defined as a general time in bed. As apparent from the above description, a data range that the data inputter 16 inputs is not limited to twenty-four-hours data, and may be, for example, seven-hours data from 11 p.m. to 6 a.m. on the next day. The dip detector 18 detects a group of dips that satisfy a predetermined dip shape from data such as dip widths and dip depths. A dip detection method will be described later in detail. The dip depth calculator 20 calculates respective depths of the dips in the dip group detected by the dip detector 18. A dip depth calculation method will be described later in detail. It should be noted, as the R-R interval measuring device, a polysomnography may be used instead of the Holter electrocardiograph. Further, pulse rate interval time series data that is measured by a plethysmograph or heart rate interval time series data that is measured by an ictometer may be used instead of the R-R interval time series data. The plethysmograph may be, for example, a wearable plethysmograph.

The heart rate variation index calculator 22 calculates an amplitude of cyclic variation of a high frequency component (0.15 Hz to 0.45 Hz) from the R-R interval time series data. The heart rate variation index calculator 22 can extract a frequency component according to one of the calculation methods described hereinbelow. For example, the heart rate variation index calculator 22 may calculate the amplitude of cyclic variation of the high frequency component using complex demodulation analysis. Further, the heart rate variation index calculator 22 may calculate the amplitude of cyclic variation of the high frequency component using fast Fourier transform or autoregression analysis. The heart rate variation index calculator 22 may calculate the amplitude of cyclic variation of the high frequency component using wavelet transform or short-time Fourier transform. The heart rate variation index calculator 22 may calculate a root mean square of a difference between consecutive R-R intervals (root mean square of successive difference) as an estimate of the amplitude of cyclic variation of the high frequency component.

The individual threshold determination processor 24 determines a threshold unique to data related to a depth of a dip, which is to be CVHR candidate, as a data-specific threshold from the amplitude of cyclic variation of the high frequency component extracted by the heart rate variation index calculator 22. In this embodiment, a value that is 2.5 times the amplitude of cyclic variation of the high frequency component is employed as the data-specific threshold. The dip width calculator 26 calculates a width for each of the plurality of local dips (that is, a duration of time during which each dip is appearing). The dip interval calculator 28 calculates intervals between respective pairs of two consecutive dips. A dip interval is a time period from a center point of a dip width of one dip to a center point of a dip width of its adjacent dip.

The dip group determiner 30 performs the respective processes as below.

(1) A group of dips having larger dip depths than the data-specific threshold is specified as a significant dip group from among the plurality of local dips.

(2) A group of dips having predetermined similar shapes is specified as a similar dip group from among the significant dip group specified in the above (1).

(3) A group of dips that are consecutive with a predetermined cyclicity is specified as a cyclic dip group from among the similar dip group specified in the above (2).

Each dip in the cyclic dip group specified in (3) is CVHR.

In the above (1), the data-specific threshold calculated for each data is used as a determination criterion for the significance of the dip depth, so the dip group specified in the above (1) will be termed the significant dip group. The dip group specified in the above (2) will be termed the similar dip group. The dip group specified in the above (3) will be termed the cyclic dip group. It should be noted that, the dip detector 18, the dip depth calculator 20, the heart rate variation index calculator 22, the individual threshold determination processor 24, the dip width calculator 26, the dip interval calculator 28, and the dip group determiner 30 correspond to an example of “CVHR detection means”.

The ACV logarithm calculator 32 averages the dips in the cyclic dip group specified by the dip group determiner 30 (waveforms of the CVHR), calculates an amplitude of cyclic variation thereof as an amplitude of cyclic variation (ACV) of heart rate, and calculates a logarithm thereof. It should be noted that the ACV logarithm calculator 32 corresponds to an example of “CVTR shape property index acquisition means”.

The FCV logarithm calculator 34 calculates a frequency of the CVHR per one hour (that is, FCV) that appear in the R-R interval time series data that is to be a processing target, and calculates a logarithm thereof. It should be noted that the FCV logarithm calculator 34 corresponds to an example of “FCV acquisition means”.

The ACV score calculator 36 corrects the ACV logarithm calculated by the ACV logarithm calculator 32 based on the FCV logarithm calculated by the FCV logarithm calculator 34, and calculates the same as a corrected ACV (ACV score). It should be noted that the ACV score calculator 36 corresponds to an example of “ACV correction means”.

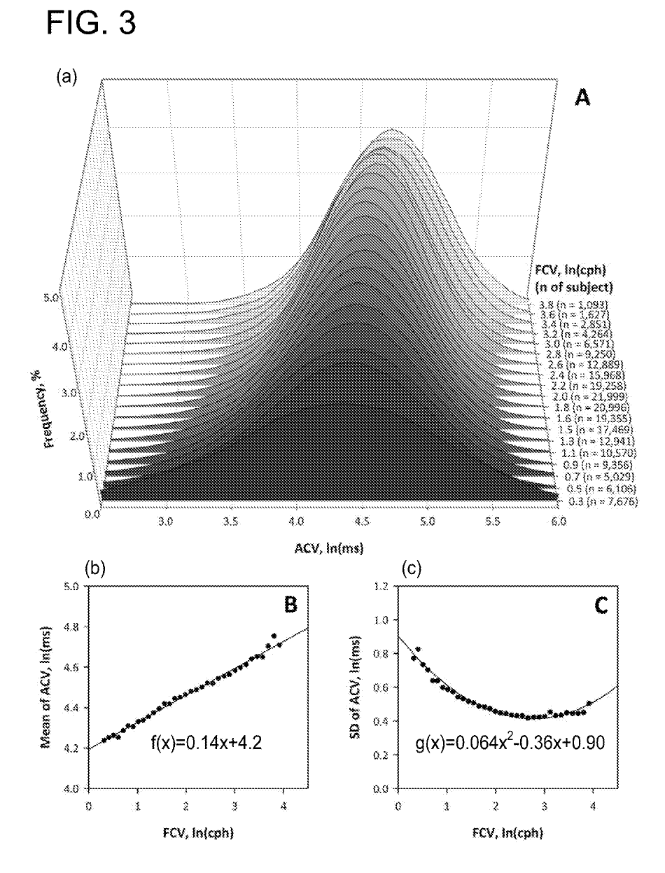

Here, a correlation relationship between FCV and ACV will be described with reference to FIGS. 3(a) to (c). A graph A in FIG. 3(a) is a graph that shows distributions of ACV natural logarithms (which may hereinbelow be simply termed ACV logarithms) for respective values of FCV natural logarithms (which may hereinbelow be simply termed FCV logarithms). The graph A is created based on a large-scale database in which data taken from Holter electrocardiograms for 210,000 subjects is accumulated. In the graph A, “n of subject” shows a number of the subjects for each FCV logarithm. A height in the graph A shows a ratio of the subjects having each ACV logarithm to the population (210,000 subjects). A graph B in FIG. 3(b) is a graph that shows a mean of the ACV logarithms in the graph A for each FCV logarithm. According to the graph B, the mean of the ACV logarithms increases linearly according to an increase in the FCV logarithm, and it can be understood that a behavior thereof is approximate to a function f(x)=0.14x+4.2 (x: FCV logarithm, f(x): ACV logarithm mean). Further, a graph C in FIG. 3(c) is a graph that shows a standard deviation (SD) of the ACV logarithm distribution in the graph A for each FCV logarithm. According to the graph C, it can be understood that a behavior of the standard deviation of the ACV logarithm is approximate to a function g(x)=0.064x²−0.36x+0.90 (x: FCV logarithm, g(x): SD of ACV logarithm).

The memory 40 (to be described later) stores the above two functions f(x) and g(x). The ACV score calculator 36 uses the functions f(x), g(x) to calculate the ACV score (to be described later).

The other processor 38 performs various processes other than the ones as aforementioned. The processes performed by the processor 38 will be described later in detail.

The memory 40 may comprise a ROM, an EEPROM, a RAM, and the like. The memory 40 stores various types of information. In this embodiment, the memory 40 stores the aforementioned two functions f(x), g(x). Further, the memory 40 stores the R-R interval time series data inputted to the R-R interval time series data inputter 16. Further, the memory 40 stores an appearance time, a width, and a depth of each dip. Further, the memory 40 stores various types of information related to the dip groups (that is, the CVHR waveforms) specified by the dip group determiner 30. Specifically, the memory 40 stores the amplitude of CVHR (ACV), the frequency of CVHR (FCV) per one hour, and the corrected ACV (ACV score). The operation unit 42 includes a plurality of keys. A user can input various types of information to respective units of the CVHR shape measurement device 2 by operating the operation unit 42. The display 44 displays various types of information on its screen.

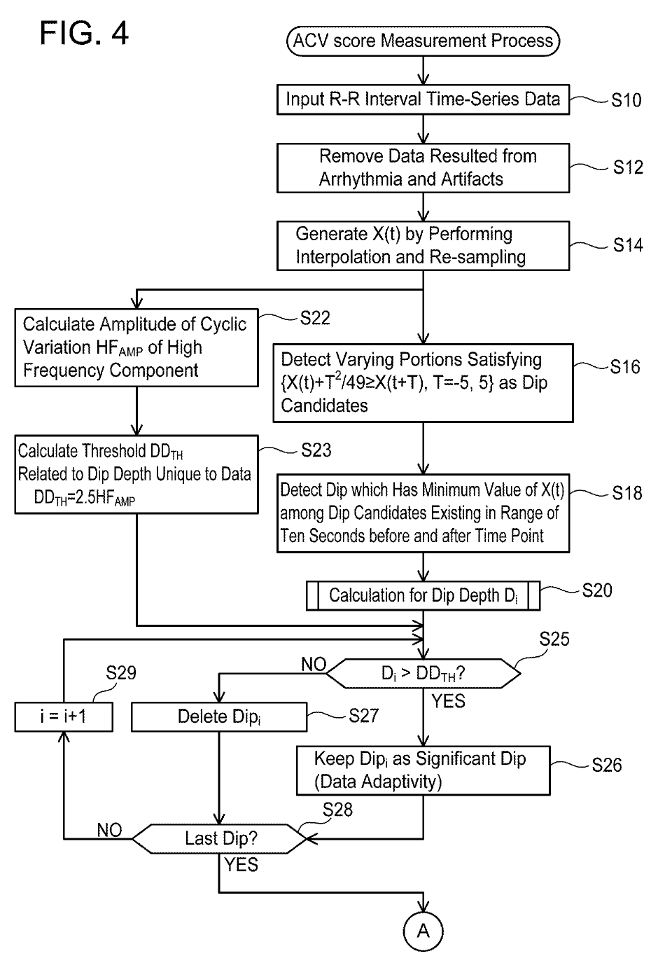

Contents of an ACV score calculation process performed by the executable code installed in the CVHR shape measurement device 2 will be described. FIGS. 4 to 9 show flowcharts of the ACV score calculation process. The R-R interval time series data inputter 16 inputs the R-R interval time series data through the communication line 14 (S10).

The R-R interval time series data inputted in S10 includes data variations resulted from nonphysiologic arrhythmia such as extrasystole or cardiac block, and artifacts. As such, the processor 38 performs a calculation process to remove the data variations resulted from the nonphysiologic arrhythmia and the artifacts (S12). Due to this, the data variations resulted from causes other than physiologic heart rate variation, and apneic and hypopnoeic heart rate variation can be removed.

In S14, the processor 38 performs an interpolation of the R-R interval time series data. For example, if a step interpolation is to be performed, an interpolation function in which its function value would take a constant value equal to a value of an R-R interval for each R-R interval is to be used. Then, the processor 38 re-samples the value of the interpolation function at 2 Hz frequency. Due to this, R-R interval time series data X(t) sampled at a regular interval is generated. Then, the dip detector 18 detects time points t, which satisfy the following (Formula 1) on the time series data X(t) for every T within a range of −5 to 5 seconds, as time points where dip candidates exist (S16).

{X(t)+T²/49≧X(t+T),T=−5,5} (Formula 1)

(Formula 1) detects varying portions to which parabolas may be inscribed as the time points where the dip candidates exist, wherein the parabolas (H=T²/49, where T is time [s] from a central axis of each parabola, and H is a height [ms] from a vertex of each parabola) have vertexes on their lower parts when the time series-data X(t) is drawn as a graph based on time t.

In a case where a vertex of a parabola inscribed to a dip candidate is smaller than any of other vertexes of parabolas inscribed to dip candidates existing in a range of ten seconds before and after the time point, the dip detector 18 specifies this dip candidate as a dip (S18). A position at which the parabola is inscribed to the dip specified by the dip detector 18 is a minimum value of that dip. Hereinbelow, the minimum value of the dip may be termed a dip bottom. Further, the time where the dip bottom exists may be termed a dip bottom time.

The dip depth calculator 20 calculates a dip depth Dᵢ for each of the plurality of local dips detected in S18. i is an ordinal of the detected dips. FIG. 7 shows a flowchart of a calculation process for the dip depth D. The dip depth calculator 20 performs the processes of FIG. 7 (S50 to S56) for each dip detected in S18.

The dip depth calculator 20 calculates moving averages for five-second frames in the time series data in a range of twenty-five seconds before and after a center time of a dip. A time series in which phase displacement of the acquired moving averages is corrected will be denoted X_{MV5}(t) (S50). X(di) is calculated at a center point (center time d) in a time axis direction of the dip (S54). X(dᵢ) is a value in a vicinity of the dip bottom. The dip depth calculator 20 calculates the dip depth Dᵢ using (Formula 2) as below (S56).

Di={max[X_{MV5}(t),t=dᵢ₋₂₅,dᵢ]+max[X_{MV5}(t),t=dᵢ,dᵢ₊₂₅]}/2−X(dᵢ) (Formula 2)

That is, the dip depth calculator 20 calculates a maximum value of the moving average X_{MV5}(t) in the twenty-five second range before the center point dᵢ of the dip and a maximum value of the moving average X_{MV5}(t) in the twenty-five second range after the center point dᵢ, and then calculates a mean value of those maximum values as a baseline value. The dip depth calculator 20 calculates the dip depth Dᵢ by calculating a difference between the baseline value and the value in the vicinity of the bottom.

In S22 of FIG. 4, the heart rate variation index calculator 22 calculates an amplitude of cyclic variation HF_{AMP} of the high frequency component (0.15 to 0.45 Hz) from the R-R interval time series data using the fast Fourier transform. The heart rate variation index calculator 22 sets a threshold DD_{TH} related to the dip depth unique to the data as a value that is 2.5 times the HF_{AMP} (S23). The amplitude of cyclic variation HF_{AMP} of the high frequency component is calculated for each data. Thus, the DD_{TH} becomes a data-specific threshold that is suitable for its corresponding data.

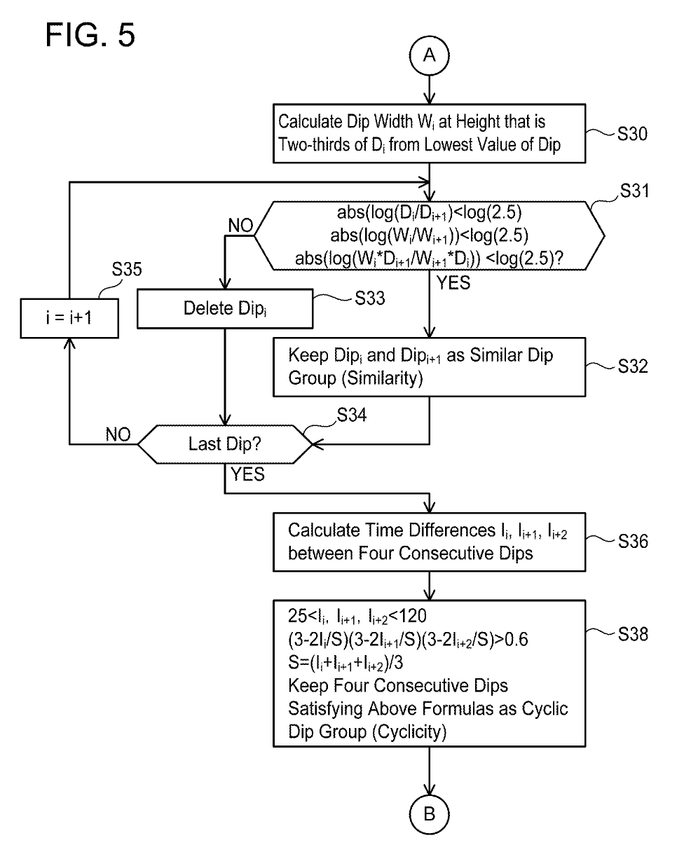

The dip group determiner 30 determines whether or not a dip i is a significant dip according to whether or not the dip depth Dᵢ is greater than the data-specific threshold DD_{TH} (S25). Here, in a case of YES, the dip group determiner 30 keeps the dip i as a significant dip (S26). A group of dips that were kept in S26 is a significant dip group. Then, the dip group determiner 30 determines whether or not this dip i is the last dip in the R-R interval time series data (S28). Here, in a case of YES, the process proceeds to S30 of FIG. 5. On the other hand, in a case of NO in S28, the dip group determiner 30 specifies a subsequent dip (S29), and returns to S25. Due to this, the dip depth Dᵢ and the data-specific threshold DD_{TH} are compared for the subsequent dip.

On the other hand, in a case of NO in S25, the dip group determiner 30 deletes the dip i (S27). Then, the dip group determiner 30 determines whether or not this dip i is the last dip in the R-R interval time series data (S28). Here, in the case of YES, the process proceeds to S30 of FIG. 5. On the other hand, in the case of NO in S28, the dip group determiner 30 specifies a subsequent dip (S29), and returns to S25.

In S30 of FIG. 5, the processor 38 calculates a dip width W, at a height that is two-thirds of Dᵢ from the dip bottom. Then, the dip group determiner 30 determines whether or not all of the following (Formula 3), (Formula 4), and (Formula 5) are satisfied for each dip (S31).

abs(log(Dᵢ/Dᵢ₊₁)<log(2.5) (Formula 3)

abs(log(Wᵢ/Wᵢ₊₁)<log(2.5) (Formula 4)

abs(log(Wᵢ*Dᵢ₊₁/Wᵢ₊₁*Dᵢ)<log(2.5) (Formula 5)

Here, the dip group determiner 30 determines whether or not shapes of consecutive dip i and dip i+1 are similar based on their dip widths and depths. In a case of YES in S31, the dip group determiner 30 keeps the dip i and the dip i+1 (S32). A group of dips kept in S32 is a similar dip group. Then, the dip group determiner 30 determines whether or not this dip i is the last dip in the R-R interval time series data (S34). Here, in a case of YES, the process proceeds to S36. On the other hand, in a case of NO in S34, the dip group determiner 30 specifies a subsequent dip (S35), and returns to S31. In S31, the dip group determiner 30 determines presence/absence of similarity for the subsequent dip.

On the other hand, in a case of NO in S31, the dip group determiner 30 deletes the dip i (S33). Then, the dip group determiner 30 determines whether or not this dip i is the last dip in the R-R interval time series data (S34). Here, in the case of YES, the process proceeds to S36. On the other hand, in the case of NO in S34, the dip group determiner 30 specifies a subsequent dip (S35), and returns to S31.

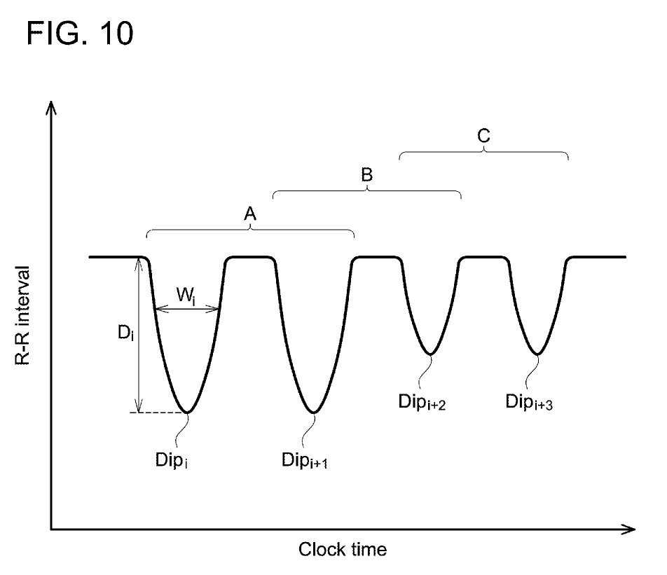

FIG. 10 is a schematic diagram of the R-R interval time series data. A determination method by which the dip group determiner 30 determines which dip should be kept after having finished the process of S31 will be described in detail using FIG. 10. Dip i to dip i+3 appear consecutively in time series. W, is the dip width of the dip i. Dᵢ is the dip depth of the dip i. Firstly, the dip group determiner 30 determines the similarity in a combination A of the dip i and the dip i+1. Then, it determines the similarity in a combination B of the dip i+1 and the dip i+2. Then, it determines the similarity in a combination C of the dip i+2 and the dip i+3.

In a case where the combination A satisfies the similarity, the dip group determiner 30 keeps both the dip i and the dip i+1. Then, in a case where the combination B also satisfies the similarity, the dip group determiner 30 keeps the dip i+1 and the dip i+2. At this occasion, the dip i+1 is kept in both processes for the combinations A and B. On the other hand, in a case where the combination B does not satisfy the similarity, only the dip i+2 is deleted. The dip i+1 that was once kept in the combination A will not be deleted regardless of the result of the combination B.

In a case where the combination B does not satisfy the similarity and the combination C satisfies the similarity, the dip group determiner 30 keeps the dip i+2 and the dip i+3. That is, the dip i+2 was deleted in the combination B, however, it is kept in the combination C.

In S36, the processor 38 calculates respective time differences Iᵢ, Iᵢ₊₁, and Iᵢ₊₂ between two adjacent dips among four consecutive dips in the dip group kept in S32. The time difference Iᵢ is a time difference between a center time dᵢ of the dip i and a center time dᵢ₊₁ of the dip i+1. The dip group determiner 30 keeps a group of four consecutive dips satisfying all of the following (Formula 6), (Formula 7), and (Formula 8) (S38).

25<Iᵢ,Iᵢ₊₁,Iᵢ₊₂<120 (Formula 6)

(3−2Iᵢ/S)(3−2Iᵢ₊₁/S)(3−2Iᵢ₊₂/S)>0.6 (Formula 7)

S=(Iᵢ+Iᵢ₊₁+Iᵢ₊₂)/3 (Formula 8)

Here, the dip group determiner 30 determines whether or not there is cyclicity in the group of the four dips configuring the time differences Iᵢ, Iᵢ₊₁, and Iᵢ₊₂ based on magnitudes of the time differences and deviations in the magnitudes of the consecutive time differences. A group of dips that are kept in S38 is a cyclic dip group. The CVHR shape measurement device 2 detects the cyclic dip group kept in S38 as the CVHR.

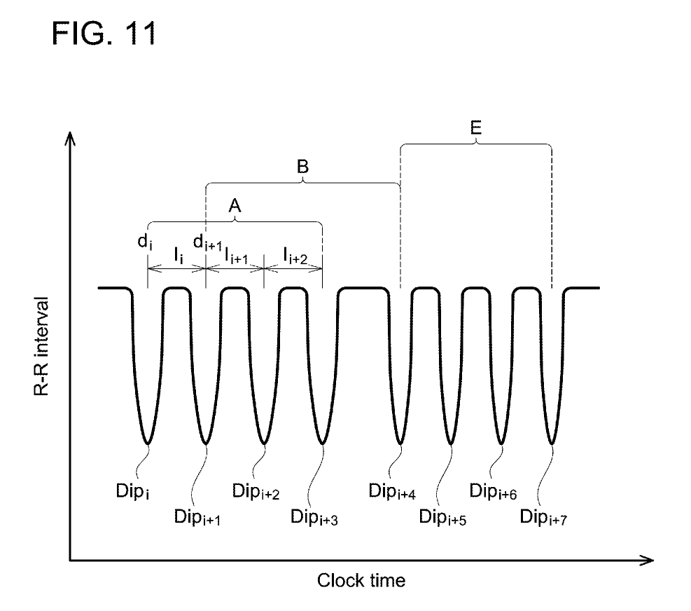

FIG. 11 is a schematic diagram of the R-R interval time series data. A determination method by which the dip group determiner 30 determines which dip group should be kept in the process of S38 will be described in detail using FIG. 11. Dip i to dip i+7 appear consecutively in time series. Firstly, the dip group determiner 30 determines the cyclicity in a combination A configured of Iᵢ to Iᵢ₊₂. Then, the dip group determiner 30 determines the cyclicity in a combination B configured of Iᵢ₊₁ to Iᵢ₊₃. The dip group determiner 30 similarly makes the determination by incrementing the dip one by one in time series order, and determines the cyclicity in a combination E configured of Lᵢ₊₄ to Iᵢ₊₆.

In a case where the combination A satisfies the cyclicity, the dip group determiner 30 keeps the dip i to the dip i+3. Then, in a case where the combination B also satisfies the cyclicity, the dip group determiner 30 keeps the dip i+1 to the dip i+4. At this occasion, the dip i+1 to the dip i+3 are kept in both processes for the combinations A and B. On the other hand, in a case where the combination B does not satisfy the cyclicity, only the dip i+4 among the dip i+1 to the dip i+4 configuring the combination B is deleted. The dip i+1 to the dip i+3 that were once kept in the combination A will not be deleted regardless of the result of the combination B.

In a case where the combination B does not satisfy the cyclicity and the combination E satisfies the cyclicity, the dip group determiner 30 keeps all of the dip i+4 to the dip i+7. That is, the dip i+4 was deleted in the combination B, however, it is kept in the combination E. Further, there are combinations that are not shown between the combinations B and E, and regardless of determination results for these combinations, the dip i+4 to the dip i+7 are kept if the combination E satisfies the cyclicity.

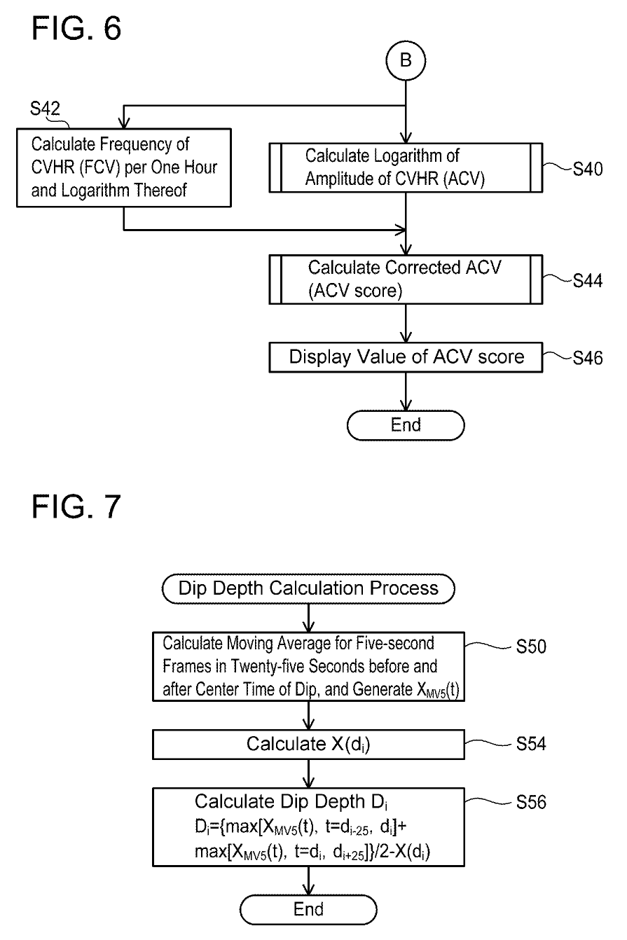

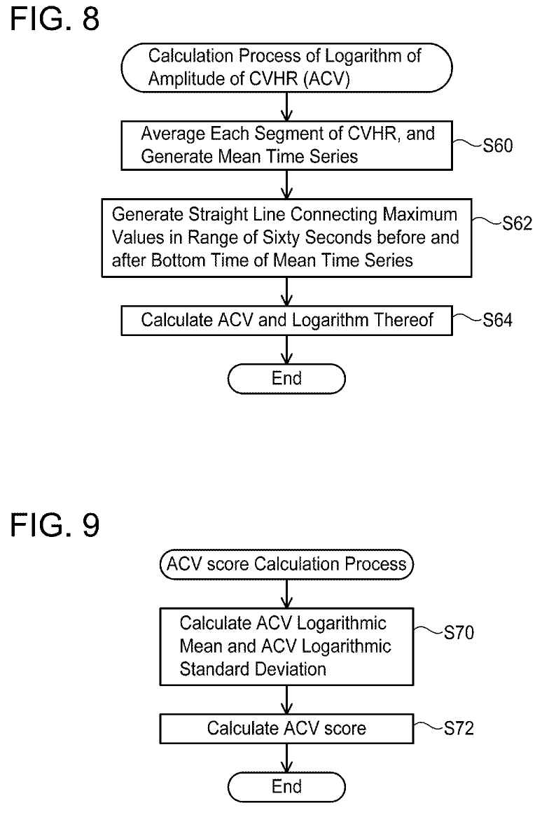

In S40 of FIG. 6, the ACV logarithm calculator 32 averages all the CVHR detected in S38, and calculates a logarithm for the amplitude (ACV) of the averaged CVHR. FIG. 8 shows a flowchart of an ACV logarithm calculation process. The ACV logarithm calculator 32 performs processes (S60 to S64) of FIG. 8.

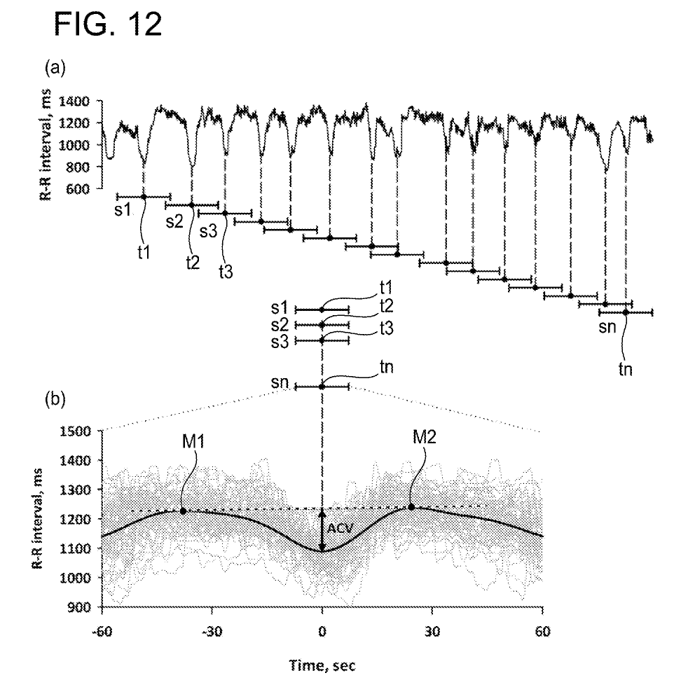

FIG. 12(a) shows n sets of the CVHR detected in S38. The ACV logarithm calculator 32 averages segments s1, s2, s3, . . . , sn which are ranges of sixty seconds before and after respective bottom times t1, t2, t3, . . . , tn of the n sets of CVHR detected in S38. Specifically, the respective segments s1 to sn are aligned with the bottom times t1 to tn of the respective CVHR as anchor points, and all of the segments s1 to sn are averaged per time. An mean time series shown by a solid line in FIG. 12(b) is thereby generated (S60). In FIG. 12(b), the anchor points of the n sets of segments s1 to sn (that is, bottom time of the mean time series) is set at a position of Time=0[s].

Next, the ACV logarithm calculator 32 creates a straight line L connecting a maximum value M1 of the mean time series in sixty seconds before the bottom time and a maximum value M2 of the mean time series in sixty seconds after the bottom time (S62) (see a broken line in FIG. 12(b)). Next, the ACV logarithm calculator 32 calculates an ACV by calculating a difference (distance) between a value of the mean time series at the bottom time and the straight line L, and calculates a logarithm thereof (S64).

In S42 of FIG. 6, the FCV logarithm calculator 34 calculates the frequency of the CVHR detected in S38 per one hour (FCV), and calculates a logarithm thereof. The FCV logarithm calculator 34 preferably calculates the FCV from the bottom time of the first CVHR to the bottom time of the last CVHR in the R-R interval time series data. The FCV may be calculated as a mean per one hour of a number of the CVHR that appear in a period from the bottom time of the first CVHR to the bottom time of the last CVHR, or may be calculated as a frequency per one hour of the CVHR within a predetermined time period.

In S44, the ACV score calculator 36 calculates the corrected ACV (ACV score). FIG. 9 shows a flowchart of an ACV score calculation process. The ACV score calculator 36 performs processes (S70 to S72) of FIG. 9.

The ACV score calculator 36 substitutes the FCV logarithm calculated in S42 to x in the two functions stored in the memory 40, namely: f(x)=0.14x+4.2 and g(x)=0.064x²−0.36x+0.90, and calculates the ACV logarithmic mean and the ACV logarithmic standard deviation (S70). Then, the ACV score calculator 36 calculates the corrected ACV (ACV score) by substituting the ACV logarithm (ln(ACV)) calculated in S40, and the ACV logarithmic mean (Mean(ln(ACV)) and the ACV logarithmic standard deviation (SD(ln(ACV))) calculated in S70 to (Formula 9) as follows (S72).

ACV score=[ln(ACV)−Mean(ln(ACV))]/SD(ln(ACV))×1.0+5.0 (Formula 9)

In S46 (see FIG. 6), the display 44 displays the ACV score calculated in S44. It should be noted that the display 44 may display a history of ACV scores, the FCV (or the logarithm thereof), the ACV logarithm calculated in S64, and/or the graph of the CVHR mean time series generated in S60, and/or the like. Further, the display 44 may display appearance times of the CVHR together with the R-R interval time series data, or may display the appearance times together with percutaneous oxygen saturation (SpO₂) or other analysis result. Further, the display 44 may display a short time period (for example, thirty minutes) within which the CVHR appearance frequency is at maximum, and the CVHR appearance frequency during that time period. Further, the CVHR shape measurement device 2 may be configured to have an audio outputter announce the ACV score instead of the display 44.

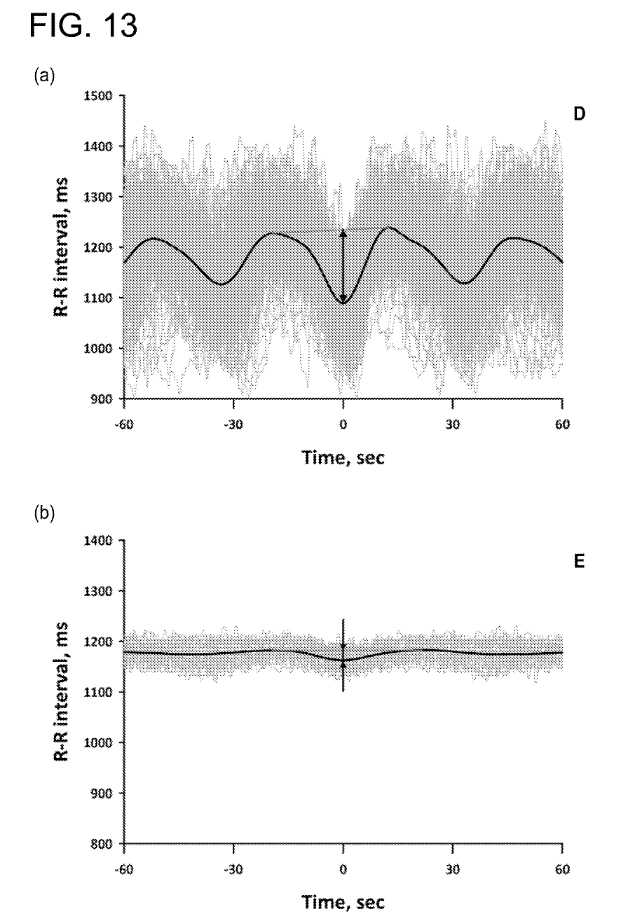

FIGS. 13(a), (b) show graphs D, E each of which shows the CVHR mean time series generated in S60. The graph D of FIG. 13(a) is an example of a subject with good prognosis, and the graph E of FIG. 13(b) is an example of a subject who passed away after one year. In comparing the graph D with the graph E, the graph D dynamically fluctuates, whereas the graph E hardly fluctuates. Due to this, the ACV of the graph D is significantly larger than the ACV of the graph E. Since the ACV is an index before the correction, the comparison of the aforementioned two cannot be said as being completely fair, however, the difference between the ACVs of the aforementioned two is evident, and it can be understood that the subject with the good prognosis has a greater ACV (that is, greater intensity of heart rate response to apnoeic load or hypopnoeic load) than the subject who passed away after a predetermined time period.

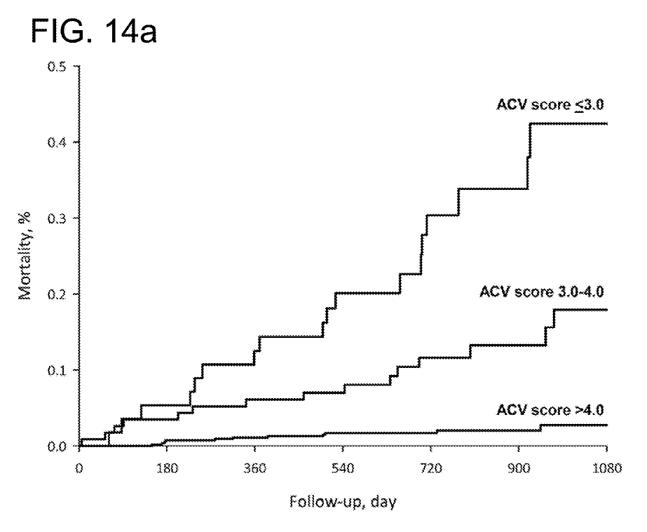

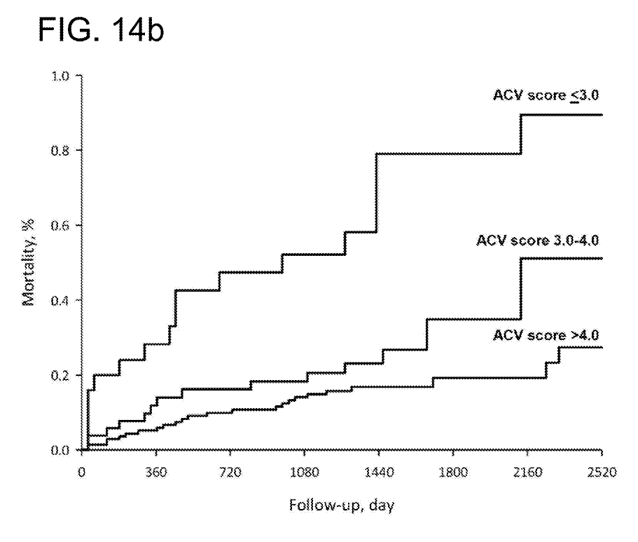

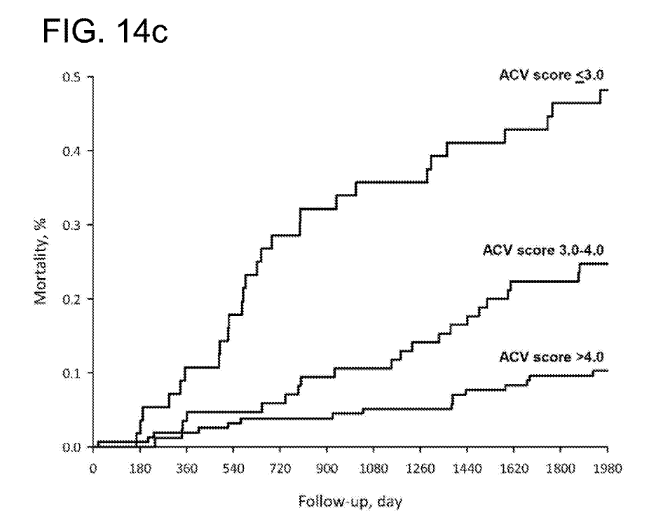

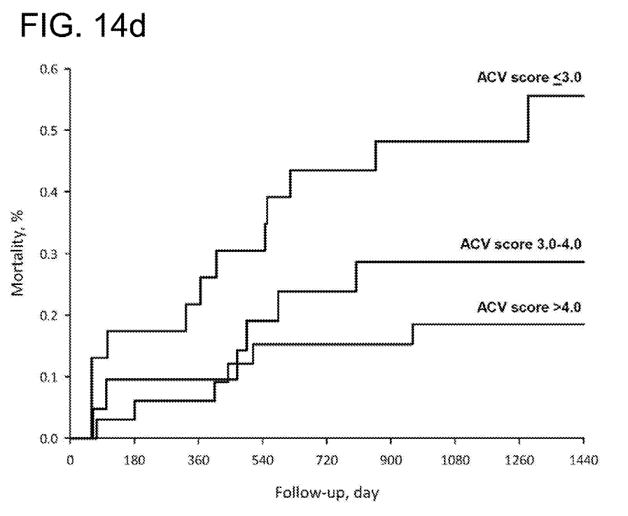

FIGS. 14(a) to 14(d) are Kaplan-Meier curves each of which indicates a relationship between the ACV score and mortality of a group being affected by a same disease or pathology. FIG. 14(a) shows the mortality of a group affected by acute myocardial infarction (n=715 people, median of follow-up period=748 days), FIG. 14(b) shows the mortality of another group affected by acute myocardial infarction (n=217, median of follow-up period=1338 days), FIG. 14(c) shows the mortality of a group of patients affected by end stage renal failure and receiving hemodialysis (n=297, median of follow-up period=2549 days), and FIG. 14(d) shows the mortality of a group affected by chronic heart failure (n=77, median of follow-up period=1172 days). In each of FIGS. 14(a) to 14(d), at least after 180 days has passed, the mortality is lower within the same time period with greater ACV score. Further, an increasing rate of the mortality is more acute with smaller ACV score, and a difference in the mortality for respective ACV scores becomes prominent as time passes. Due to this, it can be understood that the ACV score and the mortality have a strong relevance. It can be understood that the ACV score can be a strong index for predicting human mortality within a predetermined period regardless of types of diseases. Further, in each of the cases of FIGS. 14(a) to 14(d), there is a prominent difference in how the mortality increases between ACV score≦3.0 and 4.0≦ACV score. Due to this, for example, a determination can be made to perform a heart transplant with priority for a patient with heart failure having the ACV score of 3.0 or lower. Further, a determination can be made to apply an implanted cardiac defibrillator for a post-myocardial infarction patient or a patient affected by severe arrhythmia having the ACV score of 3.0 or lower. As above, values of the ACV scores can be utilized in the determination of treatment strategies for various diseases. It should be noted, although the relevance between the ACV score and the mortality was investigated in this embodiment, it has been confirmed, as a result of the study conducted by the inventor, that the ACV score also exhibits strong relevances to various health risks other than the mortality.

In the CVHR shape measurement device 2 of the present embodiment, a CVHR detection means configured of the dip detector 18 to the dip group determiner 30 detects the cyclic variation of heart rate (CVHR) from the data that indicates the heart rate of a human in time series during time in bed. The ACV logarithm calculator 32 measures the CVHR amplitude (ACV), and calculates the logarithm thereof. The magnitude of the ACV and a degree of the health risk such as the mortality during a predetermined period (health risk) are closely related. Due to this, the human health risk can be predicted more accurately than in conventional techniques by referencing the ACV measured by the CVHR shape measurement device 2.

Further, the CVHR shape measurement device 2 of the present embodiment measures the ACV by averaging the plurality of CVHR. Due to this, even in a case where respective shapes of the plurality of CVHR are different, the reliability of the ACV is increased and the ACV that more accurately reflects the intensity of the human heart rate response to the apnoeic load or hypopnoeic load during the time in bed can be acquired.

Further, as shown in FIG. 3(b), the mean of the ACV logarithm is proportional to the FCV logarithm. Due to this, even if two subjects have a same ACV, the health risk indicated by the ACV value would be different if the FCV of one subject is small and the FCV of the other subject is large. In the CVHR shape measurement device 2 of the present embodiment, the ACV score calculator 36 corrects the ACV logarithm calculated by the ACV logarithm calculator 32 based on the FCV logarithm calculated by the FCV logarithm calculator 34, and calculates the corrected ACV (ACV score). The ACV score is a generalized index independent from the FCV value. Due to this, without depending to the FCV value, the health risk of the subject can accurately be predicted by using the ACV score. Further, the health risks of subjects having greatly different FCVs can accurately be compared.

Further, in the CVHR shape measurement device 2, the ACV score calculator 36 corrects the ACV logarithm by using the two functions which are derived from the database in which the ACVs and the FCVs for respective subjects are accumulated, namely: f(x)=0.14x+4.2 (x: FCV logarithm, f(x): ACV mean) and g(x)=0.064x²−0.36x+0.90 (x: FCV logarithm, g(x): ACV logarithmic standard deviation). By using the functions derived from the database for the correction, the versatility of the ACV score can be improved. Especially, since the database of the present embodiment accumulates the ACVs and the FCVs of 210,000 subjects who are affected by various diseases such as acute myocardial infarction or end stage renal failure, use of such a database can construct an approximate function with high reliability.

Further, in the CVHR shape measurement device 2 of the present embodiment, the data measured by the Holter electrocardiograph is used as the R-R interval time series data. Due to this, there is no need for hospitalization for data acquisition as in the conventional techniques, and the data can easily be acquired. The ACV score can be acquired non-invasively, safely, and repeatedly in daily life. Due to this, by measuring the ACV score continuously and observing how values thereof shift, the CVHR shape measurement device can be used for a purpose of verifying treatment effects, or improvement effects of lifestyle habits (such as drinking and smoking) or living environment (such as PM2.5). The ACV score can be used in the medical field as an index for evaluating a health condition or as a tool for managing user's own health. Further, since a correlation between a human activity and an influence that the activitiy will impose on the heart rate can be grasped, it can be used for academic purposes (such as living standards and stress). Further, since the data can be acquired by various devices, the data collection is easy, and a database in which a large amount of data is accumulated can be constructed. An increase in the data within the database enables more detailed analysis, such as an analysis on trends according to types of diseases. As a result, the reliability and versatility of the ACV score can more easily be improved.

Further, as a result of the keen study conducted by the inventor, the health risk prediction performance of the ACV score has been revealed as being equal to or higher than the health risk prediction performance of a case of measuring the R-R intervals over twenty-four hours using the Holter electrocardiograph or the like. Due to this, if the CVHR appears even just once in the measured data, the twenty-four-hour data measurement becomes unnecessary. Especially, the CVHR shape measurement device 2 of the present embodiment uses the R-R interval time series data taken during time in bed, so there is no need to wear the Holter electrocardiograph for twenty-four hours as in the conventional techniques. Due to this, the inconvenience of wearing the Holter electrocardiograph during an active period is omitted, which allows the data to be measured more easily and more comfortably than the conventional techniques, and the health risk can be predicted at an accuracy that is equal to or higher than that predicted by the conventional techniques. Further, as a result of the inventor's analysis of the aforementioned database, it has been found that the CVHR occurs at a very high percentage, to 96.9% of men and 96.0% of women. The ACV score can be calculated if the CVHR occurs even just once. Due to this, the ACV score is an index that can be measured for almost all the subjects, and its convenience as an index is high.

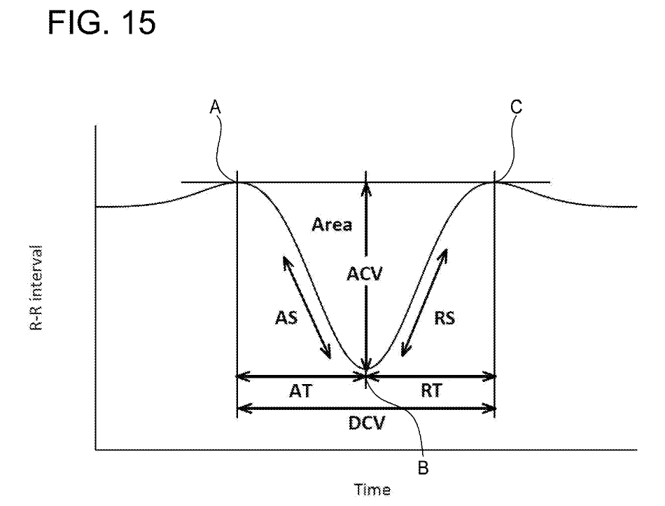

In an embodiment, the health risk was predicted using the ACV score, however, the index for predicting the health risk is not limited thereto. For example, a slope of the CVHR waveform, a ratio of the amplitude of cyclic variation to a duration of the CVHR waveform, or an area of the CVHR waveform may be used as the index. FIG. 15 shows a smoothed CVHR waveform extracted from the R-R interval time series data. The CVHR waveform includes points A, B, and C. The point B is a minimal point. The point A is a maximal point closest to the point B, and appears before the point B. The point C is a maximal point closest to the point B, and appears after the point B. An ACV is a distance between a straight line AC and the point B, an activation time AT (Activation Time) is a time that had passed from the point A to the point B, a recovery time RT (Recovery Time) is a time that had passed from the point B to the point C, and a duration DCV (Duration of Cyclic Variation) is a time that had passed from the point A to the point C. There are two types of slopes in the CVHR waveform, namely an activation slope AS (Activation Slope) and a recovery slope RS (Recovery Slope). The activation slope and the recovery slope are respectively defined as AS=ACV/AT and RS=ACV/RT. Further, the ratio of the amplitude of cyclic variation to the duration of the CVHR waveform is defined as ACV/DCV, and the area of the CVHR waveform is defined as a size of an area surrounded by the CVHR waveform and the straight line AC.

The following Tables 1 and 2 show the respective shape property indexes (indexes) of the CVHR waveform and mortality risks for respective diseases. The data indicates a hazard ratio (HR) according to Cox hazard regression analysis, 95% confidence limit (CI) thereof, χ² value, and significance probability (P). HR indicates how many times the mortality would become when each of the indexes decreases by one. The χ² value indicates accuracy of the prediction performance for the mortality risk, meaning that the prediction performance is higher with greater value.

TABLE 1

| | Post-MI 1 (acute myocardial infarction) | | | Post-MI 2 (acute myocardial infarction) | | |
|---|---|---|---|---|---|---|
| | HR (95% CI) | χ² | P | HR (95% CI) | χ² | P |
| FCV, per 1 cycle/hour increase | 1.0 (0.7-1.4) | 2.7 | 1 | 1.0 (0.7-1.3) | 0.1 | 1 |
| ACV, per 1 ln(ms) decrease | 2.6 (2.0-3.3) | 49 | <0.001 | 1.9 (1.5-2.4) | 20.7 | <0.001 |
| ACV score, per 1 decrease | 9.1 (4.6-18) | 59 | <0.001 | 2.9 (1.7-5.2) | 23.8 | <0.001 |
| AS, per 1 ms/sec decrease | 1.4 (1.2-1.5) | 28.9 | <0.001 | 1.2 (1.1-1.4) | 14.3 | <0.001 |
| RS, per 1 ms/sec decrease | 1.4 (1.2-1.5) | 28.7 | <0.001 | 1.2 (1.1-1.4) | 14.8 | <0.001 |
| ACV/DCV, per 1 ms/sec decrease | 2.5 (1.8-3.5) | 29.7 | <0.001 | 1.6 (1.2-2.2) | 10.1 | 0.001 |
| Area, per 1 sec² decrease | 3.9 (2.2-6.9) | 21.2 | <0.001 | 1.4 (0.9-2.1) | 2.3 | 0.1 |

TABLE 2

| | ESRD (end stage renal failure) | | | CHF (chronic heart failure) | | |
|---|---|---|---|---|---|---|
| | HR (95% CI) | χ² | P | HR (95% CI) | χ² | P |
| FCV, per 1 cycle/hour increase | 1.0 (0.8-1.2) | 0.2 | 0.7 | 0.8 (0.6-1.0) | 2.2 | 0.09 |
| ACV, per 1 ln(ms) decrease | 1.6 (1.3-1.9) | 16.7 | <0.001 | 1.4 (1.1-1.8) | 5 | 0.02 |
| ACV score, per 1 decrease | 2.4 (1.5-3.7) | 23.2 | <0.001 | 2.1 (1.1-4.3) | 6.7 | 0.04 |
| AS, per 1 ms/sec decrease | 1.2 (1.1-1.3) | 17 | <0.001 | 1.2 (1.0-1.5) | 5.1 | 0.02 |
| RS, per 1 ms/sec decrease | 1.2 (1.1-1.3) | 16.2 | 0.003 | 1.3 (1.1-1.5) | 6.2 | 0.01 |
| ACV/DCV, per 1 ms/sec decrease | 1.5 (1.2-1.9) | 11.5 | <0.001 | 1.9 (1.1-3.2) | 5.7 | 0.01 |
| Area, per 1 sec² decrease | 1.9 (1.2-2.9) | 7.9 | 0.005 | 1.9 (1.1-3.5) | 4.7 | 0.03 |

According to Tables 1 and 2, the significance probabilities P of the FCV are equal to or greater than 5% for all of the diseases, and thus it can be understood that there is no significant relevance with the mortality risk. On the other hand, since the significance probabilities P of the indexes other than the FCV (that is, the ACV natural logarithm, the ACV score, the activation slope AS, the recovery slope RS, the ratio ACV/DCV of the amplitude of cyclic variation to the duration, and the area (Area) are less than 5% for all of the diseases, it can be understood that they have significant relevance with the mortality risk, and thus are useful as the indexes for predicting the mortality risk. Especially, when the χ² values of the respective indexes are compared in each disease, the χ² value of the ACV score is maximum in each of the diseases. Due to this, it can be understood that the ACV score is the index that can most accurately predict the mortality risk.

The embodiments of the art disclosed herein have been described in detail above, however, these are mere examples, and the CVHR shape measurement device disclosed herein encompasses various modifications and alterations of the aforementioned embodiments. For example, in an embodiment, the data was measured using the Holter electrocardiograph, however, a device used for the data measurement is not limited thereto. For example, the data may be measured using a bedside monitor, a test device for sleep disordered breathing (CPAP device, etc.), a sensor combining a bedroom and a bed, a wristwatch-type sensor, a spectacle-type sensor, electrode-integrated clothes, a tape-type sensor to be applied to skin, or an implant-type sensor. Further, heart rate count and pulse rate count may be measured by various methods. For example, the heart rate count or the pulse rate count may be measured based on heart sound, blood vessel sound, skin temperature, body vibration, vibration at a point of center of gravity of body, or pulse wave (pressure, capacity, blood flow rate, blood amount in tissue (hemoglobin absorbance rate), biological impedance, etc.).

Further, in an embodiment, the R-R interval time series data taken during time in bed was used, however, data to be used is not limited to the data during time in bed. Data during the waking period may be used, so long as the CVHR can be detected. For example, apnea or hypopnea may take place even during the waking period in elders and heart failure patients, and as such, the CVHR may be detected during the waking period. Further, in an embodiment, data from human(s) was used, however, it is not limited to human, and data from animal(s) (more strictly, animals capable of pulmonary respiration) may be used. That is, the CVHR shape measurement device disclosed herein may target animals in general capable of pulmonary respiration, including humans.

Further, in an embodiment, the CVHR shape measurement device 2 was connected to the R-R interval measuring device via the communication line 14, however, no limitation is made to this configuration. For example, an algorithm for measuring the ACV (ACV score) may be assembled in the Holter electrocardiograph, or in a wearable plethysmograph.

Further, the CVHR detection means is not limited to the one employed in an embodiment. For example, a publicly known algorithm developed by the inventor of the present application may be used. Further, in the aforementioned CVHR detection means, at least four CVHR are detected as a group. However, an algorithm capable of detecting one CVHR may be used. Further, the CVHR shape measurement device 2 may not be provided with the display 44. For example, the CVHR shape measurement device 2 may be connected to another device, and the ACV measurement result may be outputted from this other device.

Further, the ACV calculation means is not limited to the one employed in an embodiment. For example, the ACV logarithm calculator 32 may calculate the ACV by averaging the dip depths Di, among the dip depths Di calculated by the dip depth calculator 20, of all of the CVHR detected in S38. Alternatively, the ACV logarithm calculator 32 may perform a process same as S62 on each CVHR. That is, the ACV logarithm calculator 32 may create a straight line connecting a maximum value within sixty seconds before CVHR bottom time and a maximum value within sixty seconds after the CVHR bottom time. Then, an amplitude of cyclic variation of the CVHR may be acquired by calculating a difference between the straight line and the CVHR value at the bottom time. An ACV may be calculated by performing this process on all the CVHR detected in S38, and averaging the amplitudes of cyclic variation thereof.

Further, in an embodiment, the ACV was corrected based on the FCV, however, the indexes other than the ACV (for example, AS, RS, ACV/DCV, and area) may be corrected based on the FCV. Further, these indexes may be corrected based on a factor other than the FCV (for example, CVHR width). Further, the recovery time RT of a dip may be used as an index for predicting the health risk.

Specific examples of the present disclosure have been described in detail, however, these are mere exemplary indications and thus do not limit the scope of the claims. The art described in the claims include modifications and variations of the specific examples presented above. Further, technical features described in the description and the drawings may technically be useful alone or in various combinations, and are not limited to the combinations as originally claimed. Further, the art described in the description and the drawings may concurrently achieve a plurality of aims, and technical significance thereof resides in achieving any one of such aims.

1. A device to measure a shape of cyclic variation of heart rate (CVHR), comprising:
a processor to:
detect CVHR from time series data of cardiac or pulse wave inter-beat interval, heart rate or pulse rate;
acquire at least one shape property index with respect to a waveform of the detected CVHR; and
evaluate health risk based on the at least one shape property index;
wherein the CVHR appears periodically in the time series data,
wherein the at least one shape property index is selected from the group consisting of an amplitude of the CVHR (ACV), a slope, a ratio of the ACV to a duration, and an area, and
wherein the health risk is inversely correlated with an intensity of a heart rate response determined from the at least one shape property index.

2. The device according to claim 1, wherein
the processor is to acquir at least one of the ACV, the slope, the ratio of the ACV to the duration, and the area by averaging respective waveforms of a plurality of CVHR detected during a predetermined period in the time series data.

3. The device according to claim 2, wherein:
the processor is to acquire a frequency of the CVHR (frequency of cyclic variation, FCV) per unit time which were detected during the predetermined period, and
the processor is to acquire a corrected ACV (ACV score) by correcting the ACV based on a value of the FCV.

4. The device according to claim 3, wherein the processor is to:
correct the ACV by using an average function of the ACV that is a function of the FCV and a standard deviation function of the ACV that is a function of the FCV; and
correct the acquired ACV by using a mean acquired from the average function and a standard deviation acquired from the standard deviation function which correspond to an FCV acquired from the data during the predetermined period from which the ACV is acquired,
wherein the average function and the standard deviation functions are derived from a database storing a plurality of sets of associations of an ACV acquired from the time series data during the predetermined period and an FCV acquired from the time series data during the predetermined period.

5. (canceled)

6. A device to measure a shape of cyclic variation of heart rate (CVHR) comprising:
a processor to:
input periodically appearing CVHR specified from time series data of cardiac or pulse wave inter-beat interval, heart rate, or pulse rate;
acquire at least one shape property index with respect to a waveform of the inputted CVHR; and
evaluate health risk based on the at least one shape property index;
wherein the at least one shape property index is selected from the group consisting of an amplitude of the CVHR (ACV), a slope, a ratio of the ACV to a duration, and an area, and
wherein the health risk is inversely correlated with an intensity of a heart rate response determined from the at least one shape property index.

7. A non-transitory computer-readable medium storing executable code, which, when executed by a processor of a device, causes the device to:
detect cyclic variation of heart rate (CVHR) from time series data of cardiac or pulse wave inter-beat interval, heart rate, or pulse rate;
acquire at least one shape property index with respect to a waveform of the detected CVHR; and
evaluate health risk based on the at least one acquired shape property index,
wherein the CVHR appears periodically in the time series data,
wherein the at least one shape property index is selected from the group consisting of an amplitude of the CVHR (ACV), a slope, a ratio of the ACV to a duration, and an area, and
wherein the health risk is inversely correlated with an intensity of a heart rate response determined from the at least one shape property index.

8. (canceled)
